(19) United States
(12) Patent Application Publication
(10) Pub. No.: US 2005/0019314 A1
(43) Pub. Date: Jan. 27, 2005

(54) Method and compositions for treating respiratory pathologies

(76) Inventor: Lionel Bueno, Aussonne (FR)

Correspondence Address: NIXON & VANDERHYE, PC, 1100 N GLEBE ROAD, 8TH FLOOR, ARLINGTON, VA, 22201-4714, US

(21) Appl. No.: 10/498,094

(22) PCT Filed: Dec. 20, 2002
(86) PCT No.: PCT/FR02/04506

(30) Foreign Application Priority Data
Dec. 21, 2001 (FR) 01 16706
Jul. 9, 2002 (FR) 02 08606

(51) Int. Cl. A61K 38/43

(52) U.S. Cl. 424/94.1; 514/2

(57) ABSTRACT

The present application relates to compositions and methods for treating respiratory pathologies. It equally concerns compositions and methods allowing regulation of the paracellular permeability of the pulmonary epithelium. The compositions and methods of the invention are based in particular on the use of agents or conditions modulating the tension of the cytoskeleton of pulmonary epithelial cells, particularly enterocytes. The invention may be used for preventive or curative treatment of various pathologies, such as asthma, allergies, obstructive diseases, etc., in mammals, particularly humans.

The present application relates to compositions and methods for treating respiratory pathologies. It equally concerns compositions and methods allowing regulation of the paracellular permeability of the pulmonary epithelium. The compositions and methods of the invention are based in particular on the use of agents or conditions modulating the tension of the cytoskeleton of pulmonary epithelial cells. The invention may be used for preventive or curative treatment of various pathologies, such as asthma, allergies, obstructive diseases, etc., in mammals, particularly humans.

The pulmonary epithelium is the site of very important exchanges between the external environment and the body. These exchanges can take place either across the cells of the epithelium, or by parallel systems. For instance, the transport of water or electrolytes, or yet the absorption of small molecules (molecular weight generally less than about 1000 Da) in the gastric, intestinal or colonic mucosa, takes place by the transcellular route, across epithelial cells or enterocytes. In contrast, the absorption of large molecules and the passage of toxins or immune cells occurs principally by the paracellular route, at the level of “tight junctions”, which are located between epithelial cells.

Epithelial tight junctions (or “TJ”) are linker structures between the cells lining the mucosal epithelia (gastrointestinal tract, lungs). These structures ensure and control paracellular transepithelial transport, from the exterior towards the submucosa, of various macromolecules (irritants, microorganisms). These structures also enable the migration of immune cells (e.g., immunocytes) towards the exterior. Tight junctions are flexible structures composed of a complex assembly of transmembrane proteins (occludins, claudins) and cytoplasmic proteins (zona ocludens proteins Z0-1, ZO-2, ZO-3, AF7 proteins, cingulin or 7H6, etc.), which are associated with the components of the cytoskeleton (myosin, actin filaments, etc.). Moreover, agents that disrupt actin cytoskeletal organization have been found to upregulate endothelial cell Nitric Oxide Synthase activity (WO 00/03746).

Under physiologic conditions, the degree of “partial” opening of these tight junctions permits the local immune system to be informed of the nature or “quality” of the contents of the airways.

While the intestinal epithelium and the structure of its tight junctions have been studied in the prior art, at present there are fewer data concerning the pulmonary epithelium and the functioning of its tight junctions.

The process of sensitization to allergens is a very important risk factor in the development of allergies and asthma. However, the mechanisms by which allergens initiate the phenomenon of sensitization have not been clearly documented in vivo. When allergens are inhaled, they come into contact with the pulmonary epithelial wall which prevents them from entering the body and presenting to immune cells. Nonetheless, for sensitivity to develop, this implies that certain allergens must be able to cross this epithelial barrier to interact with immune cells. The conditions under which this transfer is possible have not been clearly elucidated in vivo. Thus, while some in vitro studies in cell cultures suggest a role of tight junctions in this process, there are no in vivo data on the role of these junctions in the development of sensitization. Likewise, although Gordon et al. (Exp. Lung Res. 1998; 24: 659) suggest that taurine has a protective effect on tight junctions, there are no data establishing a correlation between tight junctions and sensitization or transfer of allergens across the pulmonary epithelium.

Experimental studies (WAN, H et al. J. Clin. Invest, 1999; 104(1): 123-33) as well as the demonstration in asthmatic subjects of a correlation between the size of extracellular spaces and the respiratory response threshold to inhaled acetylcholine (OHASHI et al. Aerugi 1990 November; 39(11): 1541-5) suggested a correlation between the degree of opening of tight junctions and the response to airborne allergens. However, these preliminary findings were not confirmed and did not give rise to new therapeutic approaches.

The present application results from the demonstration of an in vivo role of pulmonary epithelial tight junctions in the allergen sensitization process. The present application also follows from the development of new therapeutic strategies for treating respiratory pathologies, based on modulating the paracellular permeability of the pulmonary epithelium. In particular, the present application proposes, for the first time, a therapeutic approach to respiratory pathologies based on the use of compounds or conditions allowing modulation of the tension of the cytoskeleton of pulmonary epithelial cells. In particular, this approach allows control of the opening and closing of pulmonary epithelial tight junctions, without necessarily requiring de novo protein synthesis and/or significant protein and/or structural degradation in the epithelium. Such strategy allows the permeability of the pulmonary epithelium to be regulated in a specific, subtle and reactive manner, and thus to act on the transfer of allergens towards the immune cells. This strategy is especially suited to obtaining a rapid biological effect controllable over time (reversible).

In this respect, the results presented herein show that a substance able to relax epithelial tight junctions (receptor activator peptide RAP-2, LPS) promotes the accumulation of neutrophils and eosinophils in the pulmonary alveoles, as observed in bronchopulmonary disorders such as asthma. The results obtained further show that a chemical substance able to reduce the permeability of tight junctions of the pulmonary epithelium prevents the accumulation of neutrophils and eosinophils. These findings offer proof that molecules, agents, conditions or methods able to reduce or suppress the opening of tight junctions of the pulmonary alveolar or bronchial epithelium may be of value in treating pulmonary disorders, particularly those characterized by intrabronchial and alveolar accumulation of neutrophils and eosinophils, in particular asthma.

A first object of the invention is therefore based on a compound modulating the tension of the cytoskeleton of pulmonary epithelial cells, for preparing a medicament intended for the preventive or curative treatment of respiratory pathologies, preferably with the exclusion of hypoxia induced by respiratory pathologies, in particular impaired lung function. In that respect, impaired lung function can be caused by emphysema, cigarette smoking, chronic bronchitis, asthma, infection agents, pneumonitis (infectious or chemical), lupus, rheumatoid arthritis, inherited disorders such as cystic fibrosis, obesity, α₁-antitrypsin deficiency and the like. Hypoxia as used herein is defined as the decrease below normal levels of oxygen in a tissue.

Another object of the invention is directed to a method of preventive or curative treatment of respiratory pathologies, comprising administering to a subject in need of such treatment an effective quantity of a compound modulating the tension of the cytoskeleton of pulmonary epithelial cells.

The invention is thus based on the use of compounds modulating the tension and the state of contraction of the cytoskeleton of pulmonary epithelial cells. As indicated hereinabove, this approach enables control of the opening and closing of pulmonary epithelial tight junctions, without necessarily requiring de novo protein synthesis and/or significant protein and/or structural degradation in the epithelium.

The proteins composing the tight junctions are associated with the cytoskeleton of the cells they link together. It is proposed within the context of the invention that the tension of the cytoskeleton can be modulated in subjects presenting with respiratory disorders or diseases so as to act non-destructively and transiently on the permeability of their pulmonary epithelium. For example, contraction of the cytoskeleton should promote the opening of tight junctions, whereas relaxation of the cytoskeleton (or inhibition of contraction) should promote closing of the tight junctions.

Within the scope of the invention one therefore preferably uses compounds (or conditions) that modulate the contraction of the cytoskeleton of pulmonary epithelial cells (particularly human), preferably without substantially modulating the endothelial vascular and/or circulating hemodynamic permeability. Depending on the condition to be treated, one uses compounds which inhibit the contraction of the cytoskeleton of pulmonary epithelial cells, or which activate or promote it.

The activity of the compound on cytoskeletal tension may be direct or indirect, that is to say directed on the cytoskeletal components themselves or on components that regulate its tension. Although not limiting, compounds acting directly on the tension of the cytoskeleton are preferred. Furthermore, also preferred are compounds having a selective activity on the tension of the cytoskeleton, that is to say typically compounds which do not directly affect the structure of the component proteins of the tight junctions.

A compound is considered to modulate the tension of the cytoskeleton when it modulates the opening of tight junctions. Inhibition of contraction does not necessarily have to be complete or total, but contraction must be reduced sufficiently to reduce the opening of tight junctions such that the minimum decrease in paracellular permeability of the pulmonary epithelium is approximately 30%, preferably approximately 40%, even more preferably approximately 50%.

Different types of compounds may be used within the scope of the present application. Thus, according to the invention, the term “compound” must be interpreted in the broad sense, i.e. as designating any agent, substance, composition, condition, treatment or method allowing modulation of cytoskeletal tension. In an advantageous manner it is an agent (e.g. a molecule) or a combination or association of molecules.

According to a first preferred embodiment, one uses compounds which inhibit (or modulate) the contraction of the myosin light chain, or compounds which inhibit (or modulate) the degradation of actin.

An especially preferred embodiment of the invention consists in the use of compounds which inhibit (or modulate) the contraction of the myosin light chain or the degradation of actin.

In a particularly preferred embodiment, the invention is implemented by using compounds that inhibit the contraction of the myosin light chain or the degradation of actin, in particular compounds that inhibit phosphorylation of the myosin light chain.

Such compounds may be exemplified in particular by inhibitors of the myosin light chain kinase (MLCK).

A particular example of such selective (MLCK) inhibitor is compound ML-7 {1-(5-iodonaphtalène-1-sulfonyl)-1H-hexahydro-1,4-diazepine} (Makishima M. et al. FEBS Lett. 1991; 287:175). Other examples of such inhibitors can be cited such as compound ML-9 (Wilson DP. et al. J. Biol. Chem. 2001;13: 165) or other which are non selective: Wortmannin (Warashina A. Life Sci 2000;13: 2587-93), H-7 (Piao Zf et al. Mol Cell Biol Res Commun 2001;4: 307-12) et KT 7692 (Warashina A. Life Sci 2000;13: 2587-93). A particular object of the present invention is the use of compounds that inhibit MLCK selected in the group consisting of ML-7, ML-9, Wortmannin, H-7 and KT 7692, which may be alone or in combination thereof. Preferred compounds of the invention are compounds that do not present a significant or substantial effect on the vascular and/or pulmonary circulating hemodynamic permeability. In a particular embodiment, the present invention comprises the use of compounds that inhibit MLCK by excluding compounds selected in the group consisting in BDM [2,3-butanedione 2-monoxime], ML-7 [1- (5-iodonaphthalenel -sulphonyl)-1H-hexahydro-1,4-diazepine hydrochloride], ML-9 [1- (5-chloronaphthalene-1-sulfonyl)-1H-hexahydro-1,4-diazepine hydrochloride], wortmannin, H-7 [1- (5-isoquinoline sulphonyl)-2-methylpiperazine dihydro-chloride], Fasudil (HA1077) [Hexahydro 1- (5-isoquinolinesulphonyl)-1H-1,4- diazepine], W-7 [N-(6-Aminohexyl)-5-chloro-1-naphthalenesulfonamide] and A-3 [N-(6-Aminoethyl) -5-chloro-1-naphthalenesulfonamide]. Other compounds that inhibit phosphorylation of the myosin light chain can be compounds that activate the myosin phosphatase.

Other targets acting on the tension of the cytoskeleton are notably myosin binding proteins, such as for example cingulin, or junction molecules, such as cadherin-E, catenin-α or desmosomes. Modulation of the activity or the expression of these proteins permits regulation of cytoskeletal tension, within the scope of the present invention.

A specific object of the invention is therefore directed to the use of a modulator (particularly an inhibitor) of the activity or the structure or the expression of molecules of the cytoskeleton. For example, the compound may be an antisense nucleic acid, a synthetic molecule, an antibody fragment, etc.

According to another embodiment, compounds may be used which inhibit the synthesis of proteins or other molecules ensuring the link between the proteins of the cytoskeleton and the proteins of the tight junctions. Among the tight junction proteins may be cited in particular the proteins occludins, claudins, ZO-1, ZO-2, ZO-3, AF7 and 7H6. The invention provides a means of modulating the opening or closing of tight junctions which is therefore based on regulating the synthesis of linker proteins between the cytoskeleton and the proteins of the tight junctions. By stimulating such synthesis, a reinforcement of the link between tight junctions and the cytoskeleton is expected, leading to decreased permeability of the epithelium.

Other compounds that may be used in the invention comprise for example inhibitors of mitogen activated kinases (MAPKK), particularly the kinase MEK1 or kinase P13, such as compounds PD098,059 {2-(amino-3-methoxyphenyl)-4H-1-benzopyran-4-one} (Alessi et al., J. Biol. Chem. 1995; 270, 27589) or LY294002 {2-(4-morpholinyl)-sphenil-1 (4H)-benzopyran-4-one} (Vlahos et al., J. Biol. Chem. 1994; 269: 5241).

Other molecules that may be used to indirectly regulate the tension of the cytoskeleton include growth factors, such as hepatic growth factor (HGF), endothelial growth factor (EGF) or certain cytokines that can be released by immune cells, such as interleukins-1, -4, -13, or factors such as IFG-1 or gamma-interferon.

Another approach for indirectly regulating the tension of the cytoskeleton is based on the use of taurine or the peptide GLP2 (glucagon-like peptide 2) or yet derivatives thereof, which can alter pulmonary epithelial permeability through an indirect effect on cytoskeletal contraction. Similarly, certain molecules acting on the receptors located at the apical pole of epithelial cells (e.g., proteinase receptors; PAR-2) can act indirectly on the cytoskeleton.

A preferred embodiment of the invention comprises the use of agents acting directly on the tension of the cytoskeleton, particularly molecules which inhibit cytoskeletal contraction, especially molecules which inhibit the contraction of the myosin light chain, or which inhibit the degradation of actin.

As noted hereinabove, in an advantageous manner the compounds used are molecules, which may be alone or in combination, biological extracts, etc. Such molecules may be synthetic, semi-synthetic or biological, particularly of animal, viral, plant or bacterial origin.

The present invention may be used for treating or managing diseases or disorders of the respiratory system, particularly asthma, allergies, obstructive disorders (bronchitis, bronchiolitis, emphysema, etc.), especially when such pathologies are chronic or severe. It is particularly adapted to the preventive or curative treatment of asthma or various allergies (dust, pollen, pollution, etc.) as well as to the local treatment of lung inflammation. It may be used preventively in subjects with a predisposition or sensitivity to this type of disorder, or curatively, for example during attacks or over longer periods. The compositions and methods of the invention make it possible to reduce the suffering or respiratory difficulties of subjects, and attenuate the symptoms or the cause of these disorders.

A particular object of the invention is based on the use of a compound such as defined hereinabove for preparing a medicament intended to control, particularly to reduce, the paracellular permeability of the pulmonary epithelium of subjects with respiratory diseases, particularly pulmonary disorders characterized by intrabronchial and alveolar accumulation of neutrophils and eosinophils, for example asthma and allergy.

Another particular object of the invention consists in the use of a compound such as defined hereinabove for preparing a medicament intended to reduce sensitization to allergens in subjects presenting with or sensitive to respiratory diseases, particularly pulmonary disorders characterized by intrabronchial and alveolar accumulation of neutrophils or eosinophils, for example asthma or allergy.

A further particular object of the invention consists in the use of a compound such as defined hereinabove for preparing a medicament intended to reduce transepithelial migration of immune cells and accumulation of immune cells in the lungs of subjects presenting with a respiratory disease, particularly a pulmonary disorder characterized by intrabronchial and alveolar accumulation of neutrophils or eosinophils, for example asthma or allergy.

The invention equally relates to methods for treating the hereinabove conditions, comprising administering to a subject presenting with a respiratory pathology or sensitive to respiratory pathologies, a compound or treatment such as defined hereinabove. In a preferred manner, the compound or treatment is administered at an effective dose to reduce the paracellular permeability of the pulmonary epithelium and/or to reduce sensitization to allergens and/or to reduce transepithelial migration of immune cells and accumulation of immune cells in the lung.

The compound may be administered by different routes and in different forms. For example, the compound may be a liquid, solid or aerosol, typically in the form of a tablet, capsule, aerosol, ampoule or oral solution, solution for injection, etc. Compounds formulated for local administration are preferred (e.g. in the airways (e.g. respiratory) or by the oral route (oral solutions, tablets, ampoules, syrups, sprays, etc.). Aerosol packaging is especially preferred, when this is possible. Of course, other forms of administration are possible, such as injections (intradermal, subcutaneous, intramuscular, intravenous, intra-arterial, intraperitoneal, etc.), ointments, gels, suppositories, etc.

The compounds may be used alone or in combination and/or in association with other active agents, such as for example other active substances used in the treatment of respiratory diseases. Examples include β2-agonists and anticholinergics, corticosteroids, anti-leukotrienes, etc. These different agents may be used in multidrug therapy, and administered separately, in combination, spread out over time or concomitantly.

Another object of the invention is directed to a product or a pharmaceutical combination comprising at least one compound that modulates the tension of the cytoskeleton of pulmonary epithelial cells and at least one other active agent selected from among β2-agonists, anticholinergics, corticosteroids and anti-leukotrienes, in view of combined use, separate use or spread out over time.

A further object of the invention is a pharmaceutical composition comprising at least one compound that modulates the tension of the cytoskeleton of pulmonary epithelial cells according to the present invention, preferably a compound that inhibits the contraction of the myosin light chain, more preferably a compound that inhibits the phosphorylation of the myosin light chain, especially an inhibitor of MLCK or an activator of the myosin phosphatase, and a pharmaceutically acceptable excipient, said composition being formulated preferably for oral administration or inhalation. Preferably, the compound is formulated as an aerosol and contains a carrier gas, or as an oral solution.

The compound that modulates cytoskeletal tension of pulmonary epithelial cells used as the pharmaceutical active principle is employed in therapeutically effective amounts. It is understood that the administered dose may be adapted by those skilled in the art according to the subject (patient) to be treated, the pathology concerned, the method of administration, etc. The quantities or doses of the compounds administered or used in the compositions according to the invention may be determined according to their capacity to modulate the cytoskeletal tension of pulmonary epithelial cells. This capacity and therefore setting the dose to be administered may in particular be determined by the experimental protocol described in example 7.

Other aspects and advantages of the present invention will become apparent in the following examples, which are given for purposes of illustration and not by way of limitation.

### LEGENDS OF FIGURES

FIG. 1: Effect of ML-7 on the increase in pulmonary paracellular permeability to ¹²⁵I labelled human serum albumin induced by intratracheal infusion of Pseudomonas aeruginosa LPS. LPS decreases radioactivity levels measured in the bronchoalveolar lavage fluid (BAL) whereas, in comparison with controls, these levels are significantly higher in the lungs. This increase in pulmonary permeability is inhibited by pretreating the animals with ML-7. In fact, radioactivity levels in both BAL and lung in ML-7-treated animals were similar to those of controls.

FIG. 2: Western blot of the phosphorylated (p-MLC) and native (MLC) myosin light chain following treatment of cultured NCI-H292 human bronchial cells with LPS. Incubation times are shown on each blot (C=control).

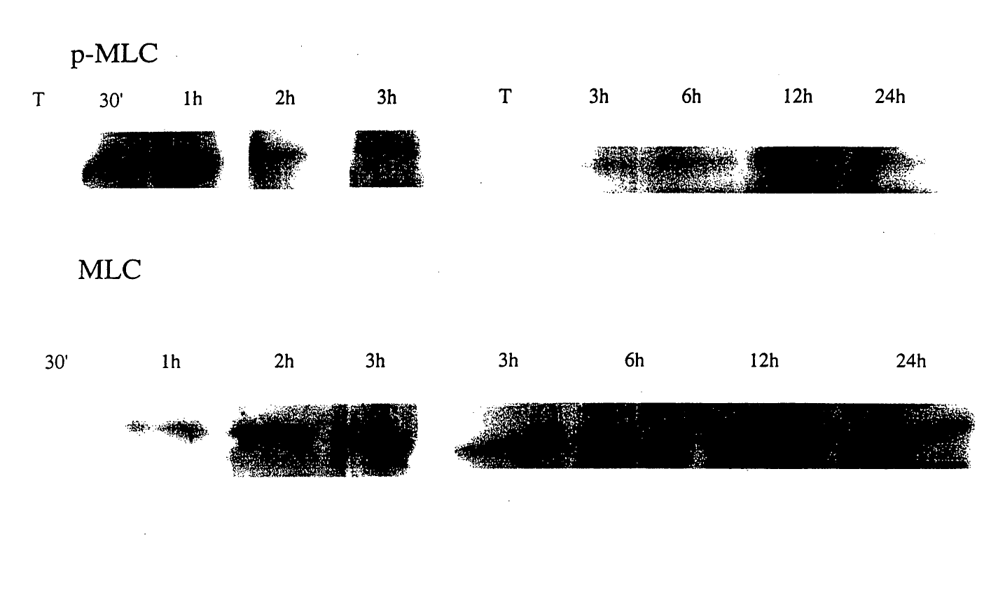

### EXAMPLES

### Example 1

### Reduction of the Bronchial Inflammatory Response by Taurine

The bronchial and alveolar epithelium possesses structures linking epithelial cells which allow controlled passage of immune cells into the airways. This example shows that certain molecules known to increase intestinal paracellular permeability such as SLIGRL promote the intra-alveolar accumulation of immune cells (neutrophils, macrophages) and that this effect can be prevented (e.g., inhibited or reduced) by oral treatment with taurine.

For this experiment, four groups of 8 male Wistar rats (250-300 g) were given drinking water containing (groups 1 and 2) or not containing (groups 3 and 4) 5% taurine, for a period of 10 days.

At time t=10 days, the four groups of animals were given a slow intranasal instillation of 200 μl of physiologic serum containing (groups 2 and 4) or not containing (groups 1 and 3) 0.2 mg of SLIGRL.

At time t=3 h after intranasal instillation, animals were anesthetized for bronchoalveolar lavage, then sacrificed.

The results are given in Table 1 below.

These results show that intranasal instillation of SLIGRL results in accumulation of eosinophils and neutrophils in bronchoalveolar lavage fluid (BAL) at t=3 h in control animals but not in animals treated with taurine (Table 1). These results provide in vivo confirmation of the role of tight junctions in the permeability of the pulmonary epithelium to immune cells.

TABLE 1
Effect of taurine on neutrophil and eosinophil accumulation in
bronchoalveolar lavage fluid induced by intranasal instillation
of SLIGRL in rats (mean ± SD; n = 10)

| PAR 2 (0.2 mg/kg IN) | | | Taurine 10% + PAR 2 | |
|---|---|---|---|---|
| (mean ± SEM | 0.9% NaCl | PAR 2 | 0.9% NaCl | PAR 2 |
| Tot. | 960 ± 112 | 6464 ± 99⁺ | 1728 ± 111 | 2086 ± 134* |
| Leucocytes | | | | |
| (mm³) | | | | |
| Macrophages | 945 ± 25 | 5559 ± 63⁺ | 1651 ± 72 | 1967 ± 103* |
| (mm³) | | | | |
| Neutrophils | 4 ± 0.3 | 656 ± 41⁺ | 34 ± 3 | 34 ± 3* |
| (mm³) | | | | |
| Eosinophils | 0.2 ± 0.7 | 32 ± 2⁺ | 17 ± 1 | 17 ± 1* |
| (mm³) | | | | |
| Lymphocytes | 144 ± 11 | 297 ± 28 | 22 ± 8 | 22 ± 8* |
| (mm³) | | | | |

*p < 0.05 from PAR 2 values;
⁺p < 0.05 from NaCl values;
IN: intranasal

### Example 2

### Reduction of the Bronchial Inflammatory Response by ML-7

This example demonstrates that ML-7 reduces the intra-aveolar accumulation of immune cells observed after an intratracheal infusion of taurocholate which induces opening of tight junctions.

For this experiment, three groups of 8 male Wistar rats (250-300 g) were given either ML-7 by the IP route at a dose of 1 mg/kg/12 h for 36 hours, or the vehicle alone. One hour after the last injection, a slow intratracheal instillation of 200 μl of physiologic serum containing (two groups) or not containing (control group) 50 mM taurocholate was given.

At time t=2 h after the intratracheal instillation, animals were anesthetized for bronchoalveolar lavage, then sacrificed.

The results are presented in Table 2 below. They show that ML-7 significantly reduces the intra-aveolar accumulation of immune cells.

TABLE 2
Effect of ML-7 on the level of accumulation of immune cells in
bronchoalveolar lavage fluid induced by intratracheal instillation
of taurocholate (5 mM/rat) (means ± SD; n = 8)

| | Control | Taurocholate | ML-7 + Taurocholate |
|---|---|---|---|
| Leucocytes | 4032 ± 919 | 35200 ± 12708* | 4160 ± 573 |
| Macrophages | 3419 ± 762 | 33288 ± 15531* | 3585 ± 398 |
| Lymphocytes | 166 ± 68 | 3654 ± 2443* | 101 ± 44 |
| Neutrophils | 445 ± 113 | 5732 ± 2279* | 473 ± 216 |

Control: 20 min intratracheal infusion of sterile water; 5 mM taurocholate intratracheal infusion: ML-7 (1 mg/kg/12 h, 36 h IP) + taurocholate
*p < 0.001 significantly different from controls

### Example 3

### Reduction of the Bronchial Inflammatory Response by PD-98059

This example shows that PD-98059 (MEK1 kinase inhibitor) reduces the intra-aveolar accumulation of immune cells associated with opening of tight junctions, induced by intratracheal infusion of taurocholate (Table 3).

For this experiment, three groups of 8 male Wistar rats (250-300 g) were given either PD-98059 by the IP route (1 mg/kg/12 h, 36 h) or the vehicle alone (DMSO). One hour after the last administration, under urethane anesthesia (25 mg/kg IP), a slow intratracheal infusion of 200 μl of physiologic serum containing (2 groups) or not containing (control group) taurocholate (5 mM/rat) was given.

Bronchoalveolar lavage (BAL) was performed two hours after the intratracheal infusion of taurocholate or the vehicle.

TABLE 3
Effect of PD-98059 on the level of accumulation of immune cells in
bronchoalveolar lavage fluid induced by intratracheal infusion of
taurocholate (5 mM/rat) (means ± SEM; n = 8).
Results expressed as number of cells/mm³ BAL.

| | Control | Taurocholate | PD-98059 + taurocholate |
|---|---|---|---|
| Leucocytes | 5040 ± 628 | 56637 ± 9791* | 21424 ± 3164*# |
| Macrophages | 4582 ± 586 | 40234 ± 5799* | 17956 ± 2465*# |
| Lymphocytes | 136 ± 31 | 4251 ± 940* | 774 ± 183*# |
| Neutrophils | 320 ± 62 | 11769 ± 5787* | 2652 ± 600*# |
| (Eosinophils) | 0 | 602 ± 173 | 27 ± 27*# |

Control: sterile 0.9% NaCl 20 min intratracheal infusion; 5 mM taurocholate per rat intratracheal infusion; PD-98059 (1 mg/kg/12 h, 36 h) + taurocholate.
*p < 0.05 significantly different from controls.
#p < 0.05 significantly different from taurocholate values.

### Example 4

### ML7 Inhibits the Increase in Pulmonary Permeability Induced by Pseudomonas aeruginosa LPS in the Rat.

This example shows that ML-7 (inhibitor of myosin light chain kinase, MLCK) significantly inhibits the increase in pulmonary permeability induced by intratracheal infusion of Pseudomonas aeruginosa lipopolysaccharide (LPS).

Pulmonary permeability was measured by means of a tracer, ¹²⁵I -labelled human serum albumin which, after intratracheal infusion, was determined in urine, plasma, lung tissue and bronchoalveolar lavage fluid.

For this experiment, three groups of 6 male Wistar rats (200-225 g) were pretreated either with ML-7 by the IP route (3 mg/kg then 1 mg/kg 3 times a day for 48 h) or the vehicle (ethanol 10%). One hour after the next-to-last administration of ML-7, under urethane anesthesia (25 mg/kg IP), a slow intratracheal infusion of 150 μl of an iso-osmolar solution (5% bovine albumin+PBS) containing the tracer and containing (2 groups) or not containing (control group) LPS (1 μg/rat) was given.

Four hours after the intratracheal infusion, urine, blood, bronchoalveolar lavage (BAL) and lungs were harvested and ¹²⁵I radioactivity was measured on each sample.

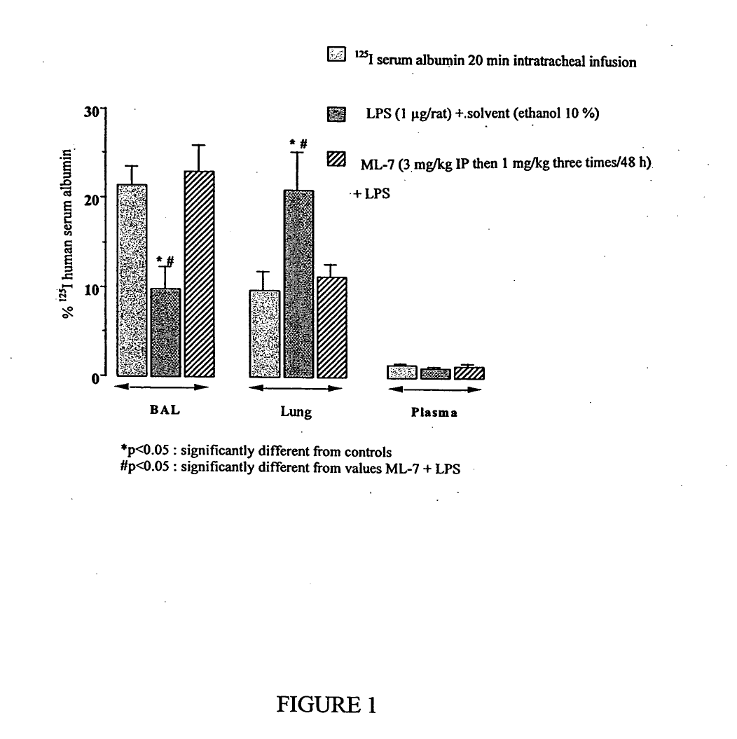

The results show that LPS decreases radioactivity measured in BAL whereas, at the same time, in comparison with controls, these levels are significantly higher in the lung. This increase in pulmonary permeability is inhibited by pretreating the animals with ML-7. In fact, radioactivity levels in ML-7-treated animals were similar to controls in both BAL and lung (FIG. 1).

In the three groups of animals, no differences in plasma radioactivity were observed.

These results show that under such experimental conditions—which can be projected to asthma and other allergic respiratory pathologies—ML-7 does not present effect on vascular endothelial cells which can modify the endothelium permeability, its tone (vasodilation) and thus generally on its pulmonary circulating hemodynamics (output, oxygenation i.e. health gain in terms of hypoxia).

Furthermore, no traces of radioactivity were detected in the urine.

In conclusion, this example shows that Pseudomonas aeruginosa LPS increases paracellular permeability in the lung, promoting the accumulation of immune cells therein and that this effect can be prevented by treatment with an inhibitor of MLCK.

### Example 5

### ML-7 Reduces the Bronchial Inflammatory Response Induced by Pseudomonas aeruginosa LPS in the Rat.

This example completes the previous example and demonstrates that ML-7 reduces the bronchial inflammatory response induced by Pseudomonas aeruginosa LPS administered by intratracheal infusion. ML-7 thereby reduces the accumulation of immune cells in the alveoles.

For this experiment, three groups of 7 male Wistar rats (200-225 g) were used. Animals were pretreated with either ML-7 by the IP route (3 mg/kg then 1 mg/kg, 3 times a day for 48 h) or with the vehicle (ethanol 10%). One hour after the next-to-last ML-7 administration, under urethane anesthesia (25 mg/kg IP), a slow intratracheal infusion of 150 μl of an iso-osmolar solution (5% bovine albumin+PBS) containing (2 groups) or not containing (control group) LPS (1 μg/rat) was given. Four hours after the intratracheal infusion, bronchoalveolar lavage was performed.

The results show that LPS increases the levels of immune cells present in BAL and more particularly that of polynuclear neutrophils. This increase in the number of neutrophils is reduced by pretreating the animals with ML-7 (Table 4).

TABLE 4
Effect of ML7 on the level of accumulation of immune cells in
bronchoalveolar lavage fluid induced by intratracheal infusion of LPS
(1 μg/rat) (means ± SEM; n = 7). Results expressed as
the number of cells/mm³ BAL.

| | Control | LPS | ML-7 + LPS |
|---|---|---|---|
| Leucocytes | 5883 ± 961 | 37968 ± 6912* | 17243 ± 2956*# |
| Macrophages | 4760 ± 738 | 16670 ± 3060* | 10708 ± 1713* |
| Lymphocytes | 188 ± 70 | 2253 ± 905* | 902 ± 375 |
| Neutrophils | 934 ± 707 | 19045 ± 4892* | 6587 ± 1749*# |

Control: 5% bovine albumin + PBS 20 min intratracheal infusion; LPS 1 μg/rat intratracheal infusion; ML-7 (3 mg/kg then 1 mg/kg 3 times a day for 48 h IP) + LPS
*p < 0.05 significantly different from controls.
#p < 0.05 significantly different from LPS values.

### Exemple 6

### LPS Stimulates Phosphorylation of Myosin Light Chain in Human Bronchial Epithelial Cells.

Materials: The human cell line NCI-H292 was obtained from the American Type Culture Collection (Manassas, Va.). Reagents, RPMI 1640 medium, fetal calf serum and other cell culture reagents were from Gibco, the protease inhibitor cocktail from Roche (1697498) and LPS (E. coli SO₅₅:B5) and other reagents from Sigma.

Cell culture: NCI-H292 cells were cultured in RPMI 1640 medium supplemented with 2 mM L-glutamine, 100 U/ml penicillin, 100 μg/ml streptomycin and 10% fetal calf serum. Cells were grown at 37° C. in a humidified 5% CO₂ atmosphere and subcultured twice a week. Cells were seeded into 6-well plates at 5.10³ cells per well. At confluence, cells were incubated in RPMI 1640 containing 0.1% bovine serum albumin (BSA) overnight. Cells were then washed with BSA-free RPMI 1640 and exposed to LPS (2 μg/ml) or physiologic serum as control (0.9% NaCl) for periods ranging from 30 min to 24 hours.

Western Blot: For this analysis, cells were lysed at different times after LPS treatment with RIPA buffer (1% Triton, 150 mM NaCl, 1 mM EDTA, 10 mM Tris pH 7.4 and protease inhibitor cocktail at the supplier's recommended concentration). The phosphorylated form of the myosin light chain (p-MLC) and the non-phosphorylated form of the myosin light chain (MLC-20) were detected in the cells at 30 min, 1, 2 and 3 hours of exposure to LPS as well as at longer periods (6, 12 and 24 hours). Proteins from LPS-treated NCI-H292 cells were separated by SDS-PAGE on a 15% polyacrylamide gel and electrophoretically transferred to a nitrocellulose membrane in 25 mM Tris-amino, 192 mM glycine and 20% methanol. Immunoprecipitation was with a goat anti-human phosphorylated myosin antibody diluted 1/500 (Santa Cruz Biotechnology Inc.) or a mouse monoclonal anti-human myosin antibody (light chains 20 K, Sigma-Aldrich, Inc.) diluted 1/1000, for detection of p-MLC and MLC-20, respectively. Peroxidase-recombinant protein G was used at 1/1000 dilution as secondary antibody. The immunolabelled bands were revealed by fluorography with ECL reagent (Enhanced chemiluminescence, Pierce, Perbio Science, Inc.).

Results: Compared to controls (physiologic serum), a maximal quantitative increase in the expression of the phosphorylated form p-MLC was observed in LPS-treated cells after 30 min and 1 h of exposure (FIG. 1), with a return to control values after 6 hours. Expression of the non-phosphorylated form MLC-20 was low after 30 min and 1 h of LPS treatment and gradually increased over time (FIG. 1: Western blots).

Conclusions: Treatment of human bronchial cells with LPS leads to a rapid increase (30 min to 1 hour) in the phosphorylation of myosin light chains which comprise the cytoskeleton of these cells. This phosphorylation reflects the contraction of the cytoskeleton and the opening of tight junctions, thereby favoring the penetration of allergens and the accumulation of immune cells in the bronchi.

### Example 7

### In Vitro Experimental Protocol for Determining the Plasma Active Concentrations of Test Compounds and Able to Act

In vitro screening of pharmaceutical formulations can be an effective and profitable method to identify the lead candidate prior to in vivo testing. Cells form tight junctions which inhibit the passage of low molecular weight substances in solution and the flow of electrical current, making it possible to use transepithelial resistance (TER) as a correlate of permeability.

Measurement of electrical resistance: Human lung cells grow to confluence on porous cell culture membrane inserts and their electrical resistance is measured with an electrical resistance device. An insert without a cell monolayer serves as control for baseline resistance and inserts with a confluent cell monolayer treated with PBS serve as controls. Pseudomonas aeruginosa LPS is added at concentrations of 1.0 ng/ml to 10 μl/ml followed by incubation for 6 hours at 37° C. Transepithelial electrical resistance (ohms×centimeter squared) is calculated by the following formula: (TERₛₐₘₚₗₑ−TER_{control})×area. Pseudomonas aeriginosa LPS decreases electrical resistance in a dose-dependent manner. The value obtained corresponding to the maximum dose inducing a response with the maximum reduction in TER is considered a 100% response and is reported for the test compounds.

Test compounds: Using the same protocol with the maximum LPS dose (100% response), the test compounds are incubated 1 hour beforehand at concentrations of 10 μM to 500 μM. The doses of test compound chosen for future tests or studies are the concentrations which cause a 50% reversion of the maximum LPS-induced decrease in TER.

This test may be employed to evaluate the 50% inhibitory effect of test compounds on the decrease in TER following LPS treatment of cell monolayers of human bronchial epithelial cells (NCI-H292).

1-12. (canceled).

13. Method for the treatment of a respiratory pathology comprising administering to a subject in need thereof a compound that inhibits the contraction of the myosin light chain of pulmonary epithelial cells.

14. Method according to claim 13, wherein the respiratory pathology is any respiratory pathology with the exception of hypoxia induced by said respiratory disease.

15. Method according to claim 13, wherein the compound inhibits the phosphorylation of the myosin light chain.

16. Method according to claim 13, wherein the compound is an inhibitor of MLCK.

17. Method according to claim 13, wherein the compound is ML-7.

18. Method according to claim 13, wherein the compound is an activator of the myosin phosphatase.

19. Method according to claim 13, wherein the compound reduces paracellular permeability of the pulmonary epithelium, without presenting a significant effect on the vascular endothelium permeability.

20. Method according to claim 13, for reducing sensitization to allergens in subjects presenting with or sensitive to respiratory diseases.

21. Method according to claim 13, for the treatment of allergies, asthma or obstructive diseases.

22. Method according to claim 13, for reducing transepithelial migration of immune cells and accumulation of immune cells in the lung of subjects with a respiratory pathology.

23. Method according to claim 13, wherein the compound is administered by the oral route or by inhalation.

24. Method according to claim 13, wherein the compound is used in association with another active agent selected in the group consisting in □2-agonists, anticholinergics, corticosteroids and anti-leukotrienes, in view of a combined use, separate use or spread out over time.
